(19) United States
(12) Patent Application Publication
(10) Pub. No.: US 2022/0184429 A1
(43) Pub. Date: Jun. 16, 2022

(54) FULL-COVER TYPE PORTABLE AIR PURIFIER

(71) Applicant: AMA TECH CORP., New Taipei City (TW)

(72) Inventor: Mao-Tu Lee, New Taipei City (TW)

(21) Appl. No.: 17/123,105

(22) Filed: Dec. 16, 2020

(51) Int. Cl. A62B 18/00; A62B 7/10; A62B 23/02; A41D 13/11

(52) U.S. Cl. CPC A62B 18/006; A62B 7/10; A41D 13/1161; A41D 13/1138; A62B 23/02

(57) ABSTRACT

A full-cover type portable air purifier includes a breathing mask provided with a first air inlet, an air cleaning device having a fan mounted in a housing thereof to drive outside air from the first air inlet into the breathing mask, an air filter set in the breathing mask, and a wearing device designed to be worn on the user's head or ears so that the breathing mask can cover the user's mouth and nose. In this way, it can filter dust, bacteria and viruses and facilitate wearing during activities.

### BACKGROUND OF THE INVENTION

### (a) Field of the Invention

The present invention relates to air purifier technology, and more particularly to a full-cover type portable air purifier that can be worn on the user's chin and cover the mouth and nose, and can continuously provide filtered clean air to form a positive pressure isolation in the mask.

### (b) Description of the Prior Art

Today's portable air purifiers are used to filter harmful substances in the air and provide users with clean air. There are many types, such as U.S. Pat. Nos. 4,233,972, 4,430,995, etc. A machine of this type mainly comprises a mask for wearing on the head, a filter blower, and an air supply pipe connected to the mask and the filter blower. However, this portable air purifier has an air supply pipe, which causes inconvenience for the user to turn the body. Another portable air purifier combines a filter blower directly with a mask, omitting the above-mentioned air supply pipe, which can make it more convenient for users to move around. However, the structural design of the filter blower directly combined with the mask often results in too many components and complex structure. Therefore, the unit price of the sale is too high and it is not easy to be accepted by consumers, and the size of the combined mask is not easy to change and cannot meet everyone's face size. Therefore, how to provide a breathing mask that is convenient to wear, has a simplified structure and a low cost, and can replace a filter blower with multiple sizes, is the subject to be solved by the present invention.

### SUMMARY OF THE INVENTION

The present invention has been accomplished under the circumstances in view. It is the main object of the present invention to provide a full-cover type portable air purifier, which can filter dust and bacteria and viruses, and can implement a combination of a breathing mask of various sizes and an air cleaning device.

It is the main object of the present invention to provide a full-cover type portable air purifier, which, through the design of a wearing device, enables the full-cover type portable air purifier to be worn on the ears or head, thereby achieving the purpose of being worn conveniently during activities.

To achieve these and other objects of the present invention, a full-cover type portable air purifier is used to cover the user's nose and mouth, comprising a breathing cover, an air cleaning device, an air filter and a wearing device. The breathing cover comprises a bottom cover used to rest on the user's chin, a front cover extending upward from a front edge of the bottom cover and used to cover the user's nose and mouth, and a first air inlet communicating between a bottom side of the bottom cover and a top side of the bottom cover and used to allow outside air to enter the front cover. The air cleaning device comprises a housing used to be combined on the bottom cover of the breathing mask to make the housing communicate with the first air inlet, an air supply port provided on the housing, and a fan arranged in the housing and used to drive outside air from the first air inlet and then out of the air supply port into the front cover. The air filter is set between a bottom surface of the housing and an upper surface of the bottom cover and used to filter the air entering from the outside. The wearing device is designed to be worn on the user's head or ears so that the front cover of the breathing mask can cover the user's mouth and nose.

Other and further benefits, advantages and features of the present invention will be understood by reference to the following specification in conjunction with the accompanying drawings, in which like reference characters denote like elements of structure.

### BRIEF DESCRIPTION OF THE DRAWINGS

FIG. 1 is an oblique front elevational view of a full-cover type portable air purifier in accordance with the present invention.

FIG. 2 is an oblique rear elevational view of a full-cover type portable air purifier in accordance with the present invention.

FIG. 3 is an exploded oblique front view of the breathing mask, the air cleaning device and the air filter.

FIG. 4 is an exploded oblique rear view of the breathing mask, the air cleaning device and the air filter.

FIG. 5 is an exploded view of the air cleaning device.

FIG. 6 is an applied view of the full-cover type portable air purifier in accordance with the present invention.

FIG. 7 is an oblique top elevational view of an alternate form of the wearing device.

FIG. 8 is an applied view of the alternate form of the full-cover type portable air purifier in accordance with the present invention.

### DETAILED DESCRIPTION OF THE PREFERRED EMBODIMENTS

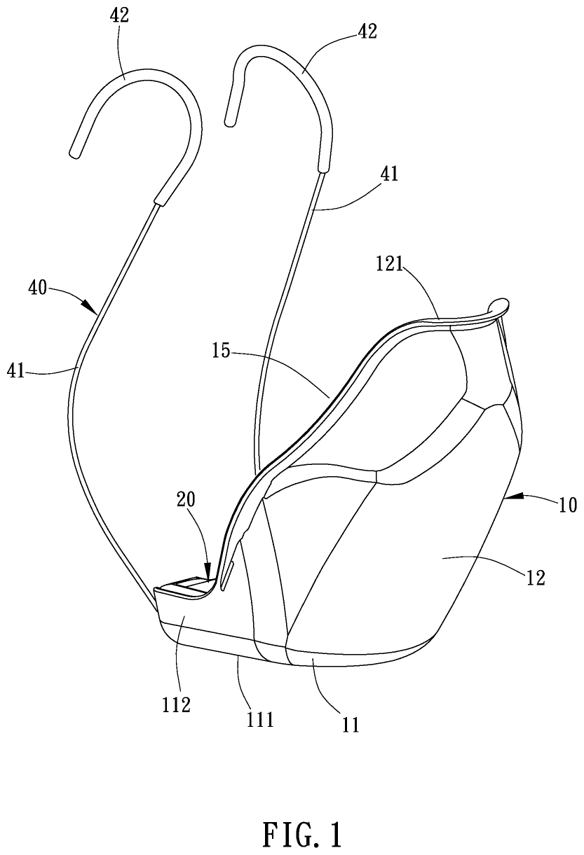

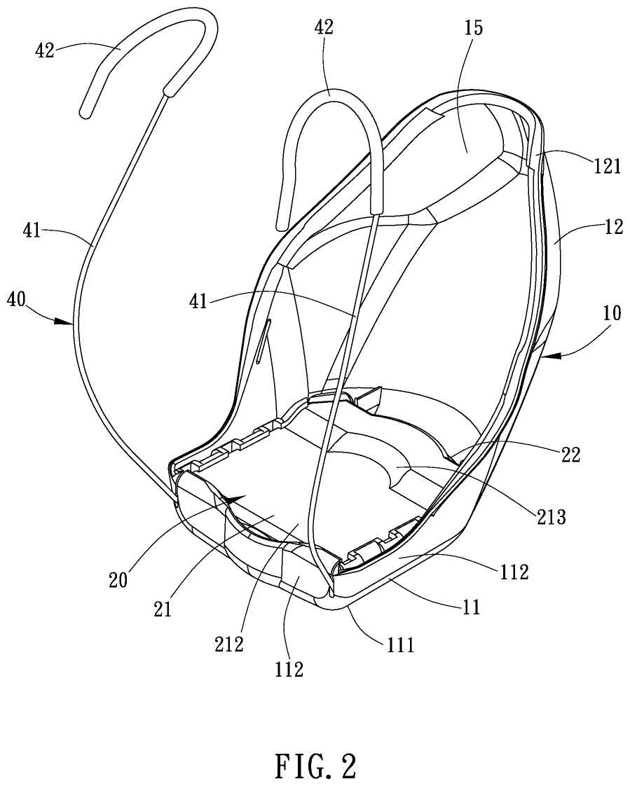

Referring to FIGS. 1 and 2, a full-cover type portable air purifier in accordance with the present invention is used to cover the user's nose and mouth. The full-cover type portable air purifier comprises a breathing mask 10, an air cleaning device 20, an air filter 30, and a wearing device 40.

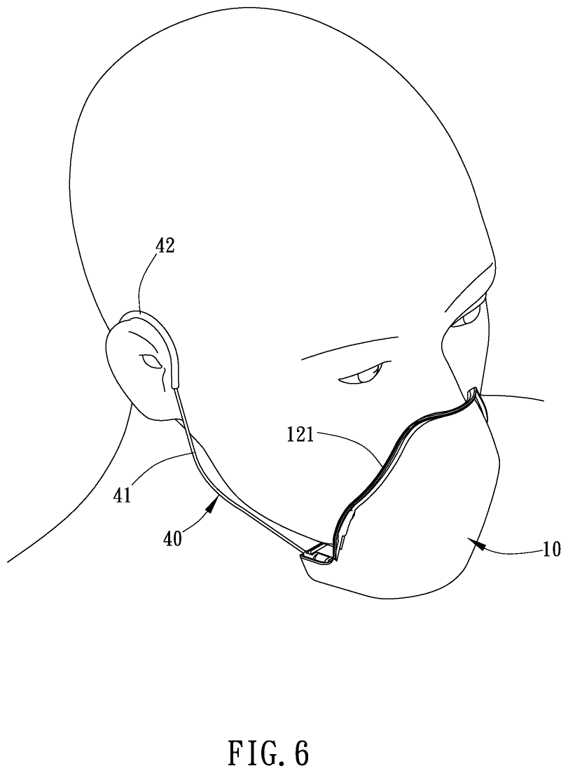

Referring to FIGS. 2-4. The breathing mask 10 is a mask made of soft plastic or silicon in one piece, comprising a bottom cover 11, a front cover 12 and a first air inlet 13. The bottom cover 11 is used to rest on the user's chin. The front cover 12 extends upward from the front edge of the bottom cover 11, so that the front cover 12 is used to cover the nose and mouth (as shown in FIG. 6). The first air inlet 13 communicates between the bottom of the bottom cover 11 and the top of the bottom cover 11, and is used to allow outside air to enter the front cover 12. The bottom cover 11 is preferably implemented with a bottom plate 111, and side walls 112 extending upward from the rear and two sides of the bottom plate 111 respectively, and then form an accommodation space 14 on the bottom plate 111. The accommodation space 14 is used to set up the air cleaning device 20 so that the air cleaning device 20 can be replaced in the accommodation space 14. The front cover 12 extends upward from the front edge of the bottom plate 111 and side walls 112, and forms a rearward opening 15 and an abutting edge 121. The abutting edge 121 is used to lean around the mouth and nose of the user. The first air inlet 13 is formed of a plurality of air holes through the bottom plate 111.

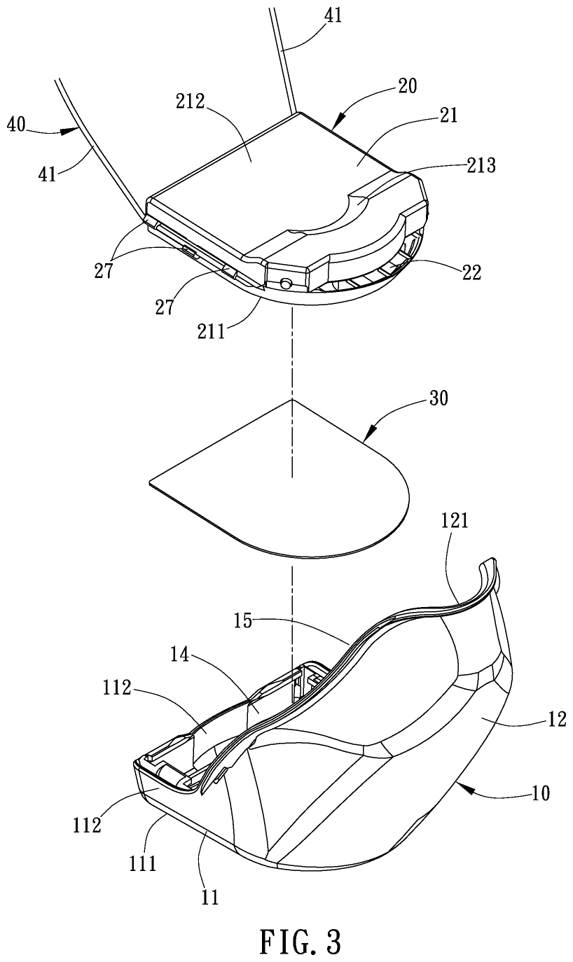

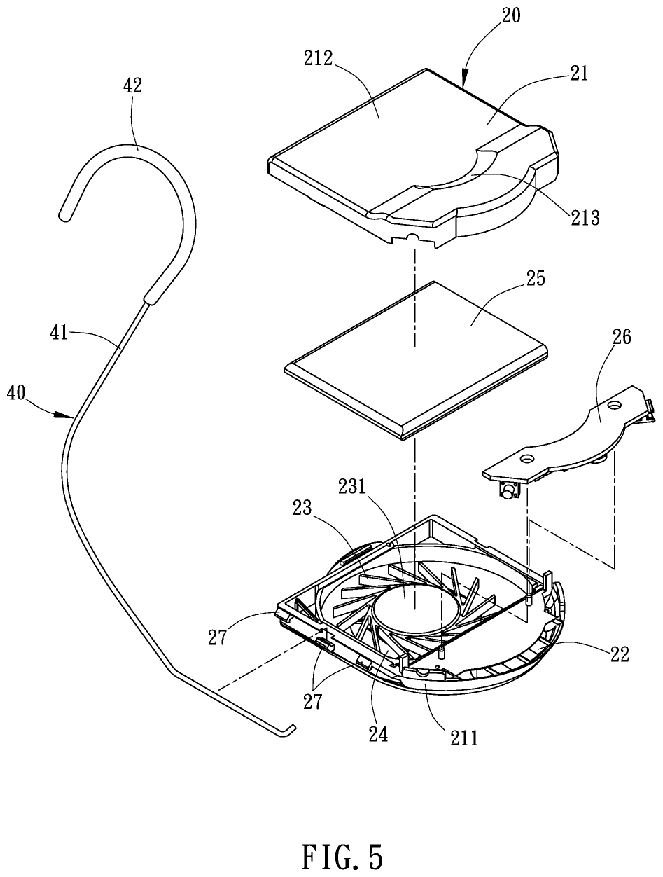

Referring to FIG. 2, FIG. 3 and FIG. 5, the air cleaning device 20 is a device used to drive outside air into the breathing mask 10, comprising a housing 21 and a fan 23. The housing 21 is used to be combined on the bottom cover 11 of the breathing mask 10 or embedded in the accommodation space 14 to make the housing 21 communicate with the first air inlet 13. An air supply port 22 is provided on the housing 21. The air supply port 22 is used to send the air sucked in from the first air inlet 13 into the front cover 12. The fan 23 is arranged in the housing 21 and can be composed of a radial fan or an axial fan. It comprises a motor (not shown) hidden in its fan hub 231, and is used to drive outside air from the first air inlet 13, and then out of the air supply port 22 into the front cover 12.

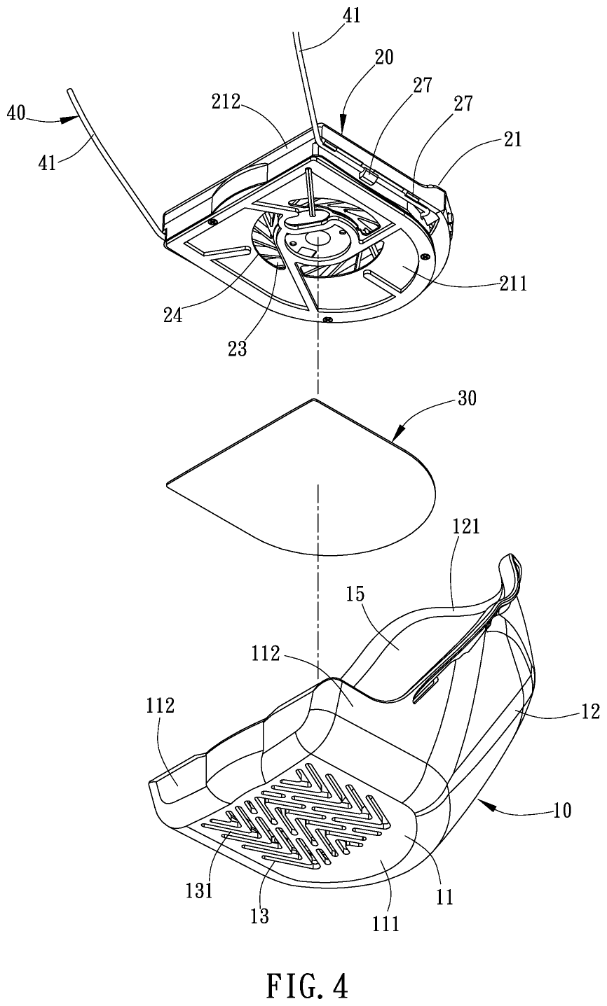

Referring to FIG. 3 and FIG. 4, the air filter 30 is a filter screen adopting electrostatic adsorption filtering method, which is set between the bottom surface of the housing 21 of the air cleaning device 20 and the upper surface of the bottom cover 11, which is convenient for the user to replate the air filter 30 when the air cleaning device 20 is removed. In other embodiments, the air filter 30 may also be disposed under the bottom cover 11, thereby being used to filter air entering from the outside.

The wearing device 40 is designed to be worn on the user's head or ears so that the front cover 12 of the breathing mask 10 can cover the mouth and nose (as shown in FIG. 6). One of the preferred embodiments of the wearing device 40 is composed of two ear-hanging strips 41. The ear-hanging strip 41 is preferably composed of a bendable shape memory alloy strip. The front end of the ear-hanging strip 41 is combined with the air cleaning device 20 or the breathing mask 10, and the rear end of the ear-hanging strap 41 has a hook 42 for hooking on the user's ear.

The above-mentioned breathing mask, air cleaning device, air filter and wearing device constitute the full-cover type portable air purifier of the present invention. Refer to FIG. 6 when using, the hooks 42 of the two ear-hanging strips 41 of the wearing device 40 are respectively hooked to the user's ears, so that the bottom cover 11 of the breathing mask 10 is leaning under the chin, and the rearward opening 15 at the back the front cover 12 covers the user's mouth and nose, and the abutting edge 121 of the front cover 12 is not completely sealed. The fan 23 of the air cleaning device 20 can be activated, so that when the fan 23 rotates, it sucks in outside air from the first air inlet 13 at the bottom of the breathing mask 10, and continuously sends the sucked air to the air supply port 22 on the housing 21, and then the sucked air enters the front cover 12 to form a positive pressure in the front cover 12 (that is, the pressure in the front cover 12 is greater than the outside air pressure). In this way, the user can breathe and prevent outside air from entering the breathing mask 10, thereby achieving the effect of filtering dust, bacteria and viruses and protecting the respiratory tract.

It is worth mentioning that the air cleaning device 20 of the present invention is a combined structure of the bottom cover 11 embedded in the integrally formed breathing mask 10, which can be used to replace the air filter 30 or select the breathing mask 10 of different sizes to achieve the effect of replacing a variety of breathing masks and an air cleaning device. In addition, by hanging the wearing device 40 on the ears, the effect of wearing during activities can also be achieved.

Referring to FIGS. 3 and 5 again, the preferred embodiment of the housing 21 of the air cleaning device 20 of the present invention comprises a bottom cover 211 and a top cover 212. The fan 23 is set between the bottom cover 211 and the top cover 212. The bottom cover 211 is provided with a second air inlet 24. When the air cleaning device 20 is embedded in the accommodation space 14 at the bottom of the breathing mask 10, the second air inlet 24 is connected to the first air inlet 13 at the bottom of the breathing mask 10. The above-mentioned air supply port 22 can be arranged at the front end of the bottom cover 211. When the top cover 212 is covered on the bottom cover 211, the top cover 212 does not cover the air supply port 22, so that the air supply port 22 is formed on the front edge of the housing 21. Alternatively, the air supply port 22 can be provided on the top cover 212 to achieve the same function and purpose.

Refer to FIG. 5 again, the air cleaning device 20 can also implement a battery 25 and a control module 26, which are respectively arranged in the housing 21. The battery 25 is a rechargeable lithium battery, which can be set in the rear part of the top cover 212. The control module 26 comprises a control circuit board and a key switch, etc., which can be embedded in the front part of the top cover 212 and connected to the fan 23 to control the start, stop and speed of the fan 23. In addition, the top surface of the top cover 212 of the housing 21 of the present invention can be implemented with a concave arc surface 213, which is used to lean against the chin of the user to achieve a stable wearing effect. The bottom cover 211 or the top cover 212 of the housing 21 can be provided with a plurality of buckling members 27 on both sides for the fastening the front ends of the ear-hanging strips 41.

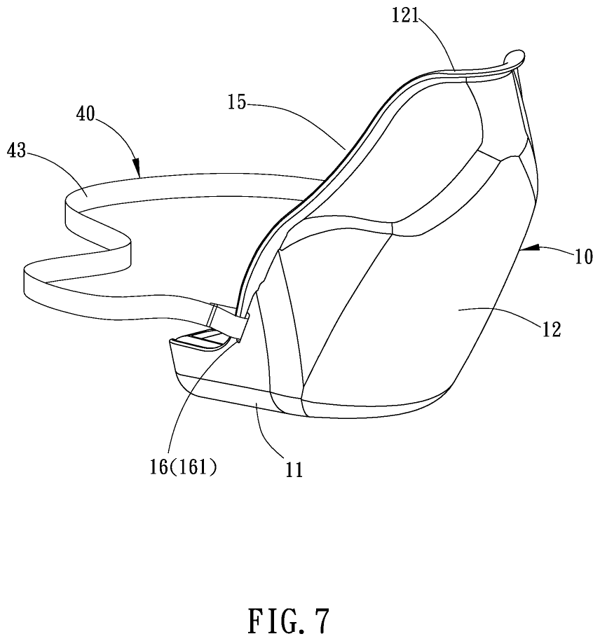

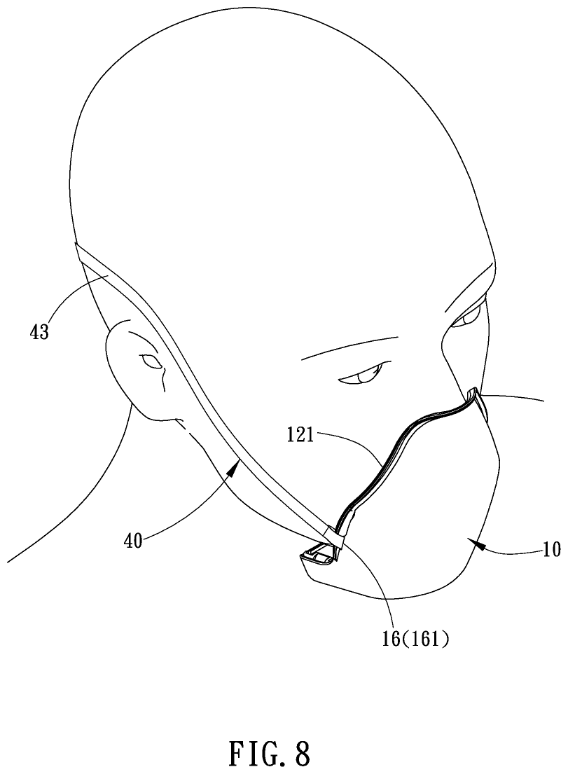

Referring to FIGS. 7 and 8, another preferred embodiment of the wearing device 40 of the present invention is a lanyard 43. The two ends of the lanyard 43 are respectively connected to the lanyard joint portions 16 provided on both sides of the breathing mask 10. The lanyard joint portions 16 can be a perforation 161 passing through the breathing mask 10, or a lug protruding from the side of the breathing mask 10. Any structure that allows the lanyard 43 to be worn and fixed can be applied in the present invention. In this way, when wearing, the lanyard 43 is wrapped around the head, and the breathing mask 10 is then covered on the nose and mouth, which can also achieve the same effect as described above.

Although a particular embodiment of the invention has been described in detail for purposes of illustration, various modifications and enhancements may be made without departing from the spirit and scope of the invention. Accordingly, the invention is not to be limited except as by the appended claims.

What is claimed is:

1. A full-cover type portable air purifier used to cover the user's nose and mouth, comprising:
a breathing cover comprising a bottom cover used to rest on the user's chin, a front cover extending upward from a front edge of said bottom cover and used to cover the user's nose and mouth, and a first air inlet communicating between a bottom side of said bottom cover and a top side of said bottom cover and used to allow outside air to enter said front cover;
an air cleaning device comprising a housing used to be combined on said bottom cover of said breathing mask to make said housing communicate with said first air inlet, an air supply port provided on said housing, and a fan arranged in said housing and used to drive outside air from said first air inlet and then out of said air supply port into said front cover;
an air filter set between a bottom surface of said housing and an upper surface of said bottom cover and used to filter the air entering from the outside; and
a wearing device designed to be worn on the user's head or ears so that said front cover of said breathing mask is capable of covering the user's mouth and nose,

2. The full-cover type portable air purifier as claimed in claim 1, wherein said housing comprises a bottom cover and a top cover, said bottom cover of said housing being provided with a second air inlet, said second air inlet being connected to said first air inlet; said air supply port is arranged at a front end of said bottom cover of said housing so that when said top cover is covered on said bottom cover of said housing, said top cover covers said bottom cover of said housing beyond said air supply port; said fan is set between said bottom cover and said top cover.

3. The full-cover type portable air purifier as claimed in claim 1, wherein said housing comprises a bottom cover and a top cover, said bottom cover of said housing being provided with a second air inlet, said second air inlet being connected to said first air inlet, said top cover being covered on said bottom cover of said housing, said top cover being provided with an air supply port; said fan is set between said bottom cover and said top cover.

4. The full-cover type portable air purifier as claimed in claim 2, wherein said air cleaning device further comprises a battery and a control module, said battery and said control module being set in said housing.

5. The full-cover type portable air purifier as claimed in claim 3, wherein said air cleaning device further comprises a battery and a control module, said battery and said control module being set in said housing.

6. The full-cover type portable air purifier as claimed in claim 1, wherein said housing comprises a concave arc surface on a top surface thereof and used to lean against the chin of the user.

7. The full-cover type portable air purifier as claimed in claim 1, wherein said bottom cover of said breathing mask comprises a bottom plate, a plurality of side walls extending upward from a rear side and two lateral sides of said bottom plate respectively, and an accommodation space surrounded by said bottom plate and said side walls; said air cleaning device is embedded in said accommodation space.

8. The full-cover type portable air purifier as claimed in claim 2, wherein said bottom cover of said breathing mask comprises a bottom plate, a plurality of side walls extending upward from a rear side and two lateral sides of said bottom plate respectively, and an accommodation space surrounded by said bottom plate and said side walls; said air cleaning device is embedded in said accommodation space.

9. The full-cover type portable air purifier as claimed in claim 3, wherein said bottom cover of said breathing mask comprises a bottom plate, a plurality of side walls extending upward from a rear side and two lateral sides of said bottom plate respectively, and an accommodation space surrounded by said bottom plate and said side walls; said air cleaning device is embedded in said accommodation space.

10. The full-cover type portable air purifier as claimed in claim 1, wherein said front cover of said breathing mask extends upward from a front edge of said bottom plate and said side walls and forms a rearward opening and an abutting edge, said abutting edge being used to lean around the mouth and nose of the user.

11. The full-cover type portable air purifier as claimed in claim 1, wherein said wearing device comprises two ear-hanging strips, each said ear-hanging strip having a front end thereof selectively combined with said air cleaning device of said breathing mask and an opposing rear end thereof provided with a hook for hooking to the user's ear.

12. The full-cover type portable air purifier as claimed in claim 1, wherein said housing of said air cleaning device is provided with buckling members for the fastening of said wearing device.

13. The full-cover type portable air purifier as claimed in claim 2, wherein said housing of said air cleaning device is provided with buckling members for the fastening of said wearing device.

14. The full-cover type portable air purifier as claimed in claim 3, wherein said housing of said air cleaning device is provided with buckling members for the fastening of said wearing device.

15. The full-cover type portable air purifier as claimed in claim 1, wherein said wearing device is a lanyard, said lanyard having two opposite ends thereof respectively connected to respective lanyard joint portions provided on both sides of said breathing mask.
